(19) United States
(12) Patent Application Publication
(10) Pub. No.: US 2014/0018888 A1
(43) Pub. Date: Jan. 16, 2014

(54) FLOW CONTROLLED RADIOFREQUENCY MEDICAL BALLOON

(71) Applicant: Boston Scientific Scimed, Inc., Maple Grove, MN (US)

(72) Inventors: Tim Ostroot, Cokato, MN (US); Derek Sutermeister, Ham Lake, MN (US); Dan Quillin, Eden Prairie, MN (US)

(21) Appl. No.: 13/927,771

(22) Filed: Jun. 26, 2013

Related U.S. Application Data
(60) Provisional application No. 61/672,095, filed on Jul. 16, 2012.
(60) Provisional application No. 61/782,154, filed on Mar. 14, 2013.

(51) Int. Cl. A61N 5/00

(52) U.S. Cl. CPC A61N 5/00
USPC 607/101

(57) ABSTRACT

A medical balloon for transmitting radiofrequency energy to a body vessel, the medical balloon comprising at least one pressurizable expanded state, the medical balloon comprising at least one electrical conductor, at least one fluid inlet and at least one fluid outlet providing a fluid flow path through the balloon, and at least one flow restrictor external to the medical balloon, wherein in the pressurizable expanded state, the balloon comprising an electrically conductive fluid circulated through the fluid flow path, the at least one electrical conductor is configured to conduct radiofrequency energy to the electrically conductive fluid and the external flow restrictor restricts fluid flow to maintain the balloon at a predetermined internal pressure.

### CROSS-REFERENCE TO RELATED APPLICATIONS

This application claims priority to U.S. Patent Provisional Application No. 61/672,095, filed Jul. 16, 2012 and U.S. Patent Provisional Application No. 61/782,154 filed Mar. 14, 2013, the entire contents of which are hereby incorporated herein by reference.

### BACKGROUND OF THE INVENTION

The present invention is directed to devices for percutaneous renal artery denervation.

Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal denervation can reduce blood pressure, and may be a viable treatment option for many patients with hypertension who do not respond to conventional drugs.

One method for treatment the renal sympathetic nerves involves a percutaneous, catheter-based therapy that uses radiofrequency energy to disrupt the renal sympathetic nerves.

One concern with this treatment is that while a desired energy or temperature is achieved at the target tissue, energies or temperatures in other portions of the artery wall may deviate enough to cause unwanted arterial tissue injury. It is thus important to maintain good contact between the device and the arterial wall and to effectively and predictably transfer heat or electrical current between the device and the arterial tissue.

For treatment of the renal artery using a catheter balloon, the balloon can be used to cool the artery to reduce injury during application of radiofrequency energy to the perivascular nerves.

Another issue with treatment using a catheter balloon is that patients have a wide range of artery sizes as well as irregularities in renal artery diameter making it difficult to accommodate a large number of patients using a commonly sized balloon catheter.

U.S. Patent Application No. 20120029509 discloses a spiral balloon catheter for renal artery denervation wherein a cooled RF balloon is configured and/or attached to the catheter's shaft in a manner that facilitates a change in the coil pitch of the balloon during inflation to accommodate varying sizes and irregularities in renal artery diameter.

There remains a need in the art for a balloon having controlled yet flexible sizing capability and that maintains consistent cooling of the artery wall during use.

### SUMMARY OF THE INVENTION

These and other aspects, embodiments and advantages of the present disclosure will become immediately apparent to those of ordinary skill in the art upon review of the Detailed Description and Claims to follow.

In one aspect the present invention relates to a medical balloon for transmitting radiofrequency energy to a body vessel, the medical balloon comprising at least one pressurizable expanded state, the medical balloon comprising at least one electrical conductor, at least one fluid inlet and at least one fluid outlet providing a fluid flow path through the balloon, and at least one flow restrictor external to the medical balloon, wherein in the pressurizable expanded state, the balloon comprising an electrically conductive fluid circulated through the fluid flow path, the at least one electrical conductor is configured to conduct radiofrequency energy to the electrically conductive fluid and the external flow restrictor restricts fluid flow to maintain the balloon at a predetermined internal pressure.

In some embodiments, the present invention relates to a medical balloon for transmitting radiofrequency energy to a body vessel, the medical balloon comprising at least one pressurizable expanded state, the medical balloon comprising at least one electrical conductor, at least one fluid inlet and at least one fluid outlet providing a fluid flow path through the balloon, and at least one flow restrictor external to the medical balloon and proximal the fluid outlet, wherein in the pressurizable expanded state, the balloon comprising an electrically conductive fluid circulated through the fluid flow path, the at least one electrical conductor is configured to conduct radiofrequency energy to the electrically conductive fluid and the external flow restrictor restricts fluid flow to maintain the balloon at a predetermined internal pressure.

In another aspect, the present invention relates to a method for controlling the size of an expandable radiofrequency medical balloon in its expanded state, the medical balloon comprising a fluid inlet and a fluid outlet providing a fluid flow path through the balloon, the method comprising the steps of providing an electrically conductive inflation fluid to the balloon through the fluid inlet, providing a flow restrictor at said fluid outlet of said expandable radiofrequency medical balloon, wherein said flow restrictor is configured and arranged to maintain said expandable radiofrequency medical balloon at a predetermined diameter.

### BRIEF DESCRIPTION OF THE DRAWINGS

FIG. 1 is a side view of an embodiment of a renal artery denervation catheter with internal parts exposed.

FIG. 1A is an enlarged view of the distal end of the catheter assembly taken at section 1A in FIG. 1.

FIG. 2A is side view of a renal artery denervation balloon formed from a based polymer material and masked.

FIG. 2B is a side view of the balloon of FIG. 2A with an outer layer of a second polymer material and masking removed.

FIG. 3 is cross-sectional view taken at section 3-3 of FIG. 1.

FIG. 4 is a schematic representation of a renal artery denervation catheter system in use according to the invention.

FIG. 5 is a perspective view of one type of a flow restrictor shown proximal the catheter system fluid outlet.

### DETAILED DESCRIPTION OF THE INVENTION

While embodiments of the present disclosure may take many forms, there are described in detail herein specific embodiments of the present disclosure. This description is an exemplification of the principles of the present disclosure and is not intended to limit the disclosure to the particular embodiments illustrated.

In some embodiments, the present invention relates to a catheter balloon configured and arranged for renal artery denervation using radiofrequency energy to disrupt the hyperactive renal nerves.

The radiofrequency (RF) balloon employs a known flow-rate of a conductive fluid that is continually circulated into and out of the balloon for conducting RF energy as well as for inflation and cooling of the balloon during use.

In alternative embodiments, other sources of energy such as ultrasound energy, microwave energy or direct heating elements may be employed for renal artery denervation.

In one aspect, the present invention relates to a radiofrequency (RF) balloon having controlled balloon sizing through the use of a flow restrictor placed in the fluid flow path of the balloon which also functions to more consistently cool the balloon.

In some embodiments, the use of an adjustable flow restrictor employed at pre-determined settings or various non-adjustable flow restrictors of known settings, allows the physician to select multiple balloon sizes within its normal operating range. For example, in the renal artery, the normal operating range of the balloon is between 4-8 mm.

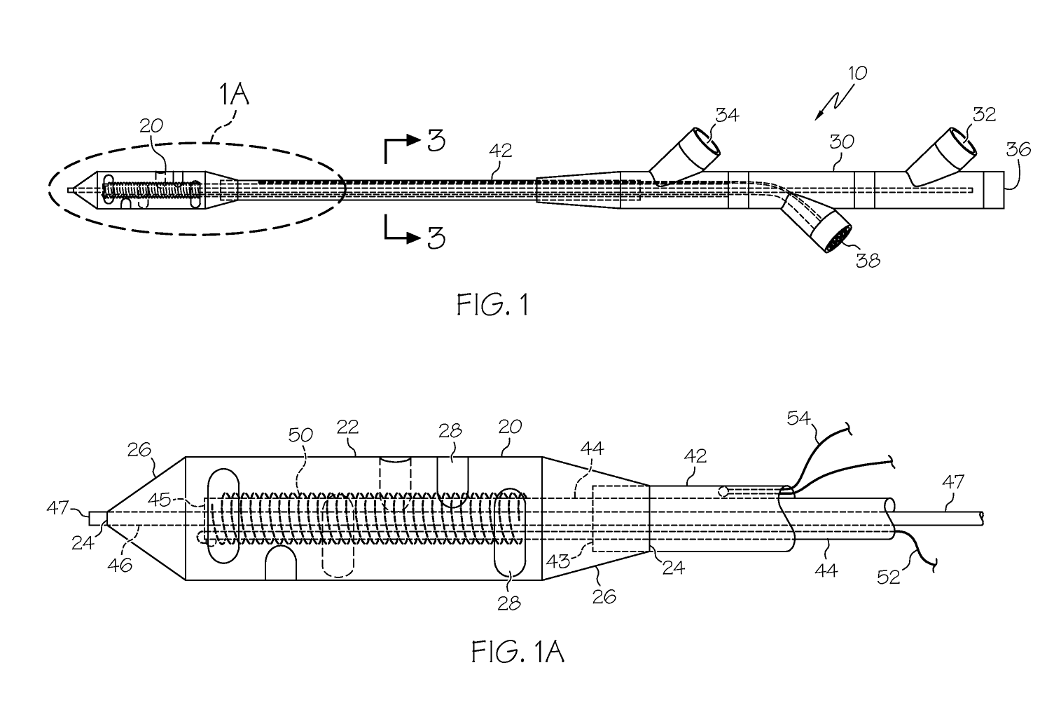

Turning now to the drawings, FIG. 1 is a side view of an embodiment of a catheter 10 comprising a renal artery denervation balloon 20 which may be employed in accordance with the invention. Catheter 10 includes a manifold 30 which in this embodiment has an inflow port 32 and outflow port 34, a guidewire port 36 and a thermocouple and power jack port 38.

As shown in FIG. 1A, taken at section 1A in FIG. 1, in this embodiment, catheter 10 has tri-shaft arrangement including an outer shaft 42, an intermediate shaft 44 and an inner shaft 46. The outer shaft 42 comprises the outflow lumen 43, the intermediate shaft 44 comprises in the inflow lumen 45 and inner shaft 46 comprises the guidewire lumen 47.

Other catheter configurations may be utilized herein and the invention is not limited as such.

Catheter 10 further includes a renal artery denervation balloon 20. Balloon 20 is expandable from a deflated configuration by supplying conductive inflation fluid via inflow port 32 through the inflow lumen 45 such that the pressure within the balloon is about 0.25 atmosphere to about 5 atmospheres, suitably about 0.5 atmosphere to about 3 atmospheres, and more suitably about 0.5 atmosphere to about 2 atmospheres. In some embodiments, the pressure within the balloon may be about 1 atmosphere or less or about 0.25 atmosphere to about 1 atmosphere.

Balloon 20 can be formed from any suitable polymeric material that allows conductivity through the balloon wall.

In some embodiments, the balloon is formed from a hydratable polymer material. Examples of hydratable polymer materials include, but are not limited to, poly(ether-block-amide materials such as PEBAX® MV1074 and MH1657 commercially available from Arkema headquartered in King of Prussia, Pa., and polyurethanes such as those that are commercially available from Lubrizol Corp. in Wickliffe, Ohio under the tradename of Tecophilic® such as Tecophilic® SG-60D-60.

In some embodiments, the hydratable polymer is blended with a non-hydratable polymer, for example, a non-hydratable poly(ether-block-amide) commercially available from Arkema under the tradename of PEBAX® such as PEBAX® 7033 and 7233, non-hydratable polyurethanes, and styrenic block copolymers such as styrene-isoprene-styrene.

The electrically insulating layer can be formed of any suitable non-conductive polymer material. Examples include, but are not limited to, hompolymeric and copolymeric polyurethanes such as those available from NeoResins Inc. in Wilmington, Mass. under the tradename of Neorez such as NeoRez R-967 and those available from Lubrizol Corp. in Wickliffe, Ohio under the tradename of Tecoflex®.

These lists of polymer materials are intended for illustrative purposes only, and not as a limitation on the scope of the present invention. Substitution of other hydratable and non-hydratable polymer materials is within the purview of those of ordinary skill in the art.

Balloons of this type are disclosed in commonly assigned U.S. Pat. No. 7,736,362, the entire content of which is incorporated by reference herein in its entirety.

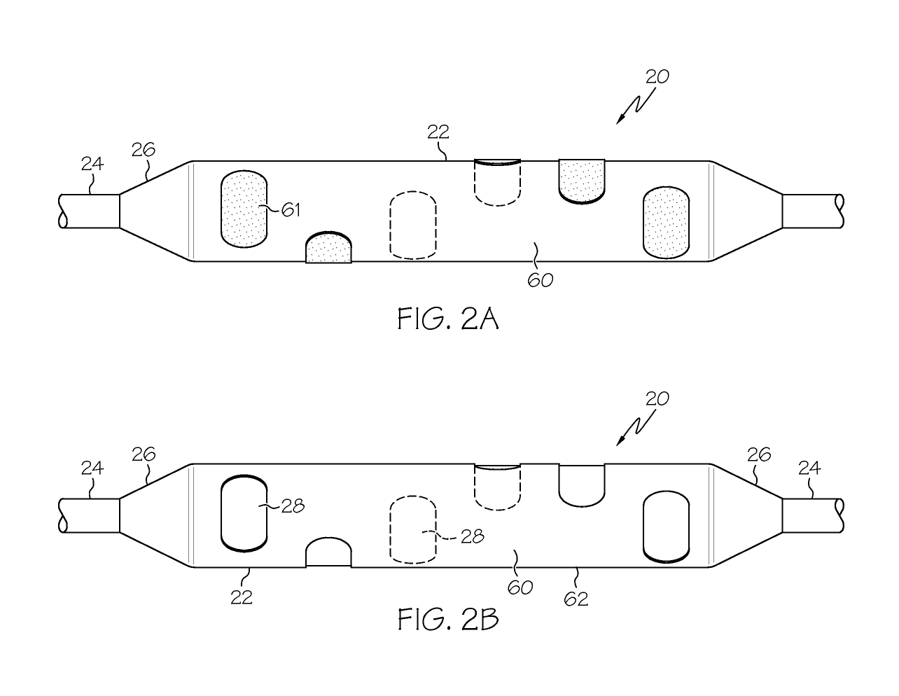

In some embodiments balloon 20 comprises a multilayer structure as illustrated in FIGS. 2A and 2B including one hydratable layer and one insulating layer. It is surmised that the hydratable layer enables ionic conduction.

FIG. 2A illustrates a first base layer 60 comprising a hydratable polymer material. Masking 61 is placed in a predetermined pattern about the first base layer 60. A second outer layer 62 comprising a non-hydratable polymer is disposed over the base layer. The non-hydratable polymer is suitably hydrophobic and insulating to the RF energy. The masking is then removed as shown in FIG. 2B leaving windows 28 wherein the first base polymer layer 60 comprising the hydratable polymer material is exposed.

Balloon 20 comprises a body portion 22 and waist and cone portions 24, 26. In this embodiment, body portion 22 comprises the windows 28.

Balloon 20 is secured at its distal waist portion 24 to the catheter inner shaft 46 and is secured at its proximal end to the catheter outer shaft 42.

Disposed within balloon 20 as shown in FIG. 1, is an electrically conductive metallic band 50 which is further fixedly connected to a power wire 52 which is disposed within lumen 45 of intermediate shaft 44. The metallic band 50 can be formed from any suitable conductive metal. In one embodiment, a silver coated copper band is employed. In another embodiment, a gold band is employed. The invention is not limited by the type of conductive metal employed for making the conductive metallic band 50.

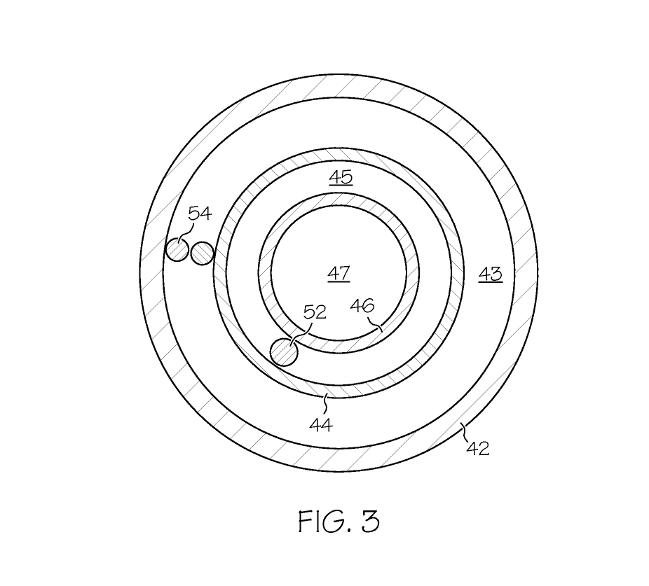

Catheter 10 further includes a thermocouple 54 disposed within lumen 43 of outer shaft 42 for accurate temperature measurement. Power wire 52 and thermocouple 54 are shown in FIG. 3 which is a cross-sectional view taken at section 3-3 in FIG. 1. Balloon 20 is inflated using an electrically conductive inflation fluid. Upon activation using RF energy, the balloon 20 is activated to provide a low RF energy to the renal artery.

A known flow-rate of a conductive fluid, such as normal saline, is continually circulated into and out of the balloon during a radiofrequency energy treatment cycle for conducting the RF energy as well as for inflating the balloon and for cooling.

It is desirable to be able to control and adjust the volumetric pressure inside of the balloon thereby controlling balloon sizing.

In embodiments according to the present invention, the balloon sizing is controlled by controlling the resistance to flow in the circulating fluid path. This can be accomplished by placing a flow restrictor proximal the outlet 34 of the fluid flow path as shown in FIG. 1.

Any suitable flow restrictor can be employed herein. The flow restrictor may be fixed or adjustable, and may consist of a means to control the pressure or a means to control the area proximal the fluid outlet 34 of the fluid flow path.

Examples of flow restrictors include, but are not limited to, spring loaded flow restrictors, screw adjusted flow restrictors, as well as tapered lumen style flow restrictors that utilize a mandrel therein.

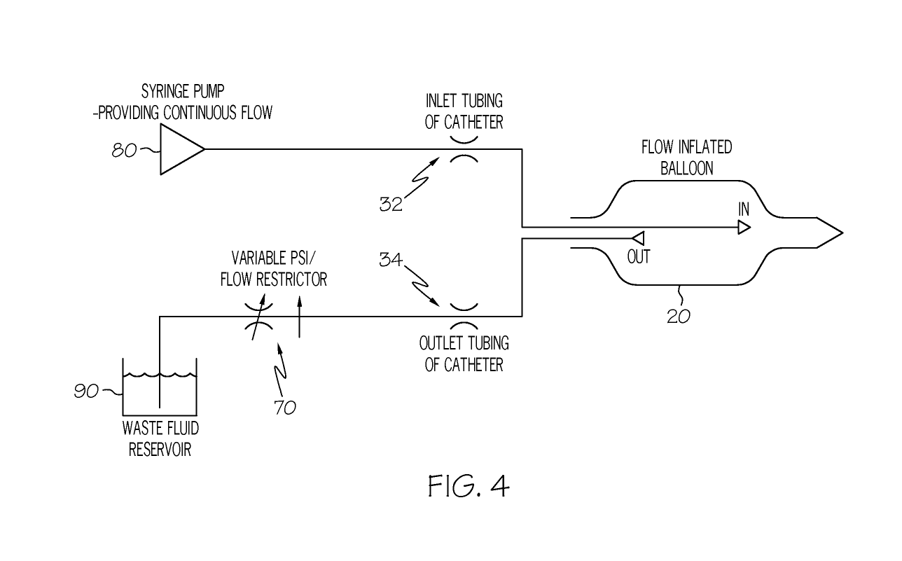

In one embodiment, as shown schematically in FIG. 4, a variable pressure flow restrictor 70 is placed proximal to the outlet 34. This provides a more consistent volumetric pressure inside of the balloon. Conductive inflation fluid is injected at inlet 32 by any suitable means such as a syringe pump 80 and provides a continuous flow of fluid into the system. The fluid exits the system via the outlet 34 into a waste fluid reservoir 90.

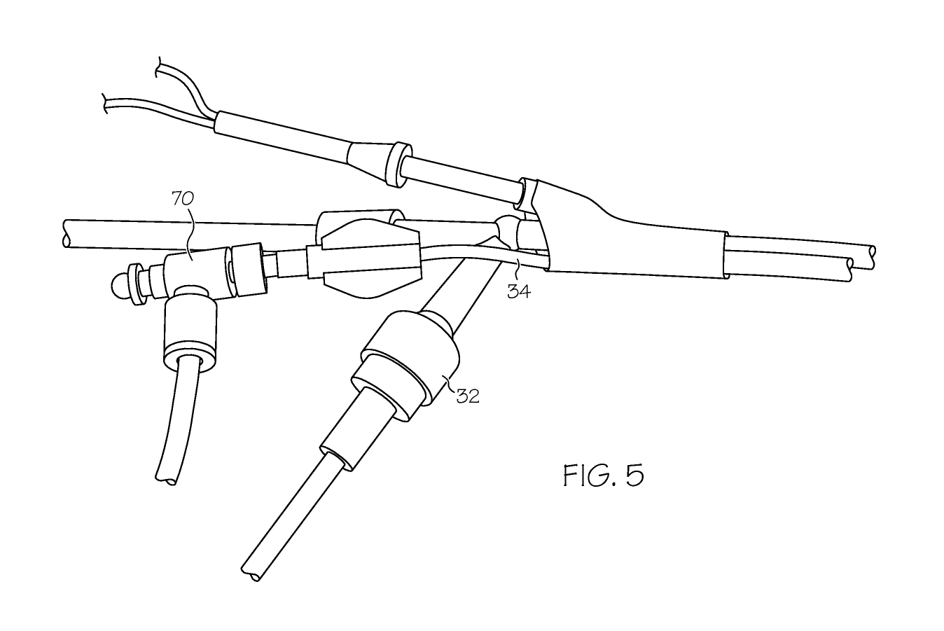

FIG. 5 illustrates a specific type of flow restrictor 70 referred to as a T-pressure relief valve. These are spring loaded flow restrictors and are commercially available from Qosina Corp. Suitable models are the 2.5 psi spring loaded flow restrictor, the 8.0 psi spring loaded flow restrictor and the 30.0 psi flow restrictor.

A suitable screw adjusted flow restrictor also referred to as a meter out flow control valve is commercially available from SMC Corporation of America, the U.S. Subsidiary of SMC Corporation based in Japan.

Multiple flow restrictors may also be employed. For example, if a volumetric pressure of about 7.5 psi (or about 0.5 atmosphere) is desirable, three 2.5 psi spring loaded flow restrictors can be employed in series.

The present invention thus allows for adjustable balloon sizing to insure good arterial wall contact for an effective denervation treatment.

While the specific embodiments disclosed herein relate to renal artery denervation, the present invention is not limited as such and can be employed with other types of RF balloons wherein the sizing of the balloon may be somewhat different.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

All published documents, including all US patent documents and US patent publications mentioned anywhere in this application are hereby expressly incorporated herein by reference in their entirety. Any copending patent applications, mentioned anywhere in this application are also hereby expressly incorporated herein by reference in their entirety. Citation or discussion of a reference herein shall not be construed as an admission that such is prior art.

1. A medical balloon for transmitting radiofrequency energy to a body vessel, the medical balloon comprising at least one pressurizable expanded state, the medical balloon comprising:
at least one electrical conductor,
at least one fluid inlet and at least one fluid outlet providing a fluid flow path through the balloon, and
at least one flow restrictor external to the medical balloon,
wherein in the pressurizable expanded state, the balloon comprising an electrically conductive fluid circulated through the fluid flow path, the at least one electrical conductor is configured to conduct radiofrequency energy to the electrically conductive fluid and the external flow restrictor restricts fluid flow to maintain the balloon at a predetermined internal pressure.

2. The medical balloon of claim 1 wherein said external flow restrictor is proximal the fluid outlet.

3. The medical balloon of claim 1 wherein said external flow restrictor is fixed or adjustable.

4. The medical balloon of claim 1 wherein said external flow restrictor is adjustable.

5. The medical balloon of claim 1 wherein said external flow restrictor comprises at least one of a means to control the pressure or a means to control the area proximal the fluid outlet.

6. The medical balloon of claim 5 wherein said external flow restrictor comprises at least one of a means to control the pressure or a means to control the area proximal the fluid outlet.

7. The medical balloon of claim 6 wherein said external flow restrictor comprises a means to control the pressure proximal the fluid outlet.

8. The medical balloon of claim 1 wherein said external flow restrictor comprises a member selected from the group consisting of spring loaded flow restrictors, screw adjusted flow restrictors and a tapered lumen flow restrictor comprising a mandrel.

9. The medical balloon of claim 1 comprising wherein said balloon is disposed about a catheter assembly having a distal end and a proximal end, the catheter shaft comprising an inner shaft, an intermediate shaft and an outer shaft, the inner shaft comprising a guide wire lumen, the intermediate shaft comprising an inflow lumen and the outer shaft comprising an outflow lumen, the inner shaft, intermediate shaft and outer shaft each having a distal end and a proximal end.

10. The medical balloon of claim 9 wherein said balloon comprises a distal waist portion, a distal cone portion, a body portion, a proximal waist portion and a proximal cone portion, the distal waist portion is fixedly disposed about the distal end of the inner catheter shaft and the proximal waist portion is fixedly disposed about the distal end of the outer shaft.

11. The medical balloon of claim 9 wherein said proximal end of said catheter assembly comprising a manifold, the manifold comprising said flow restrictor.

12. The medical balloon of claim 1 wherein said at least one electrical conductor comprises a power wire having a distal end, the distal end of the power wire is fixed to a conductive metallic band having a larger surface area than said power wire, said conductive metallic band is disposed within said balloon.

13. The medical balloon of claim 1 wherein said medical balloon comprises at least one base layer, said base layer comprises a hydratable poly(ether-block-amide), a blend of a hydratable poly(ether-block-amide) and a non-hydratable poly(ether-block-amide) or a blend of a hydratable poly(ether-block-amide) and a polyurethane.

14. The medical balloon of claim 13 wherein said medical balloon blend comprises a non-hydratable outer layer.

15. The medical balloon of claim 14 wherein said non-hydratable outer layer comprises a polyurethane.

16. The medical balloon of claim 1 wherein said balloon comprises windows formed in the outer layer.

17. A medical balloon for transmitting radiofrequency energy to a body vessel, the medical balloon comprising at least one pressurizable expanded state, the medical balloon comprising:
at least one electrical conductor,
at least one fluid inlet and at least one fluid outlet providing a fluid flow path through the balloon, and
at least one flow restrictor external to the medical balloon and proximal the fluid outlet,
wherein in the pressurizable expanded state, the balloon comprising an electrically conductive fluid circulated through the fluid flow path, the at least one electrical conductor is configured to conduct radiofrequency energy to the electrically conductive fluid and the external flow restrictor restricts fluid flow to maintain the balloon at a predetermined internal pressure.

18. A method for controlling the size of an expandable radiofrequency medical balloon in its expanded state, the medical balloon comprising a fluid inlet and a fluid outlet providing a fluid flow path through the balloon, the method comprising the steps of:
providing an electrically conductive inflation fluid to the balloon through the fluid inlet,
providing a flow restrictor at said fluid outlet of said expandable radiofrequency medical balloon, wherein said flow restrictor is configured and arranged to maintain said expandable radiofrequency medical balloon at a predetermined diameter of about 4 mm to about 8 mm.

19. The method of claim 18 wherein said flow restrictor is configured and arranged to maintain a volumetric pressure of about 3 atmospheres or less within the balloon.

20. The method of claim 18 wherein said flow restrictor is configured and arranged to maintain a volumetric pressure of about 0.25 atmospheres to about 1 atmosphere within the balloon.
